(19) United States
(12) Patent Application Publication
(10) Pub. No.: US 2017/0305823 A1
(43) Pub. Date: Oct. 26, 2017

(54) PROCESS FOR RECLAIMING ALCOHOLS

(71) Applicant: SHELL OIL COMPANY, Houston, TX (US)

(72) Inventors: Kai Jürgen FISCHER, Amsterdam (NL); Pieter HUIZENGA, Amsterdam (NL); Wouter KOOT, Amsterdam (NL); Martin MADERA, Amsterdam (NL)

(21) Appl. No.: 15/523,618

(22) PCT Filed: Nov. 2, 2015
(86) PCT No.: PCT/US2015/058609

§ 371 Date: May 1, 2017

Related U.S. Application Data
(60) Provisional application No. 62/074,992, filed on Nov. 4, 2014.

(51) Int. Cl. C07C 29/86; B01D 11/04; C07C 41/38

(52) U.S. Cl. CPC C07C 29/86; C07C 41/38; B01D 11/0488; B01D 11/0492

(57) ABSTRACT

A method of recovering an alcohol from an aqueous stream comprising: providing an aqueous stream comprising an alcohol; extracting at least a portion of the alcohol from the aqueous stream with a solvent to form an extracted solvent stream; extracting at least a portion of the solvent from the extracted solvent stream to form an extracted aqueous stream; and recovering at least a portion of the alcohol from the extracted aqueous stream.

### CROSS REFERENCE TO RELATED APPLICATIONS

This application claims the benefit of U.S. Provisional Application No. 62/074,992, filed Nov. 4, 2014, which is incorporated herein by reference.

### BACKGROUND

The present disclosure relates generally to methods of recovering alcohols from aqueous solutions. More specifically, in certain embodiments, the present disclosure relates to solvents useful for extracting alcohols from aqueous solutions and associated methods.

The extraction of hydrocarbons from deepwater oil and gas reservoirs may require the transportation of production streams from reservoirs to facilities for processing. Water, along with oil and gas, may be included in these production streams. During transportation, if the temperature of the production stream is sufficiently low and the pressure is sufficiently high, the system can enter the hydrate region where gas hydrates form. Gas hydrates are solids and, if formed, may plug the pipelines used to transport the production streams. Hydrates may also plug or cause malfunction of other units, such as valves, chokes, separators, heat exchangers, etc.

Several methods exist for limiting the formation of these hydrates in production streams. One such method is to add a hydrate inhibitor to the production streams. An example of such a hydrate inhibitor is monoethylene glycol. Another example of such a hydrate inhibitor is methanol. The hydrate inhibitors added to the production streams may later be recovered from the production stream and recycled.

Although there are several methods for recovering monoethylene glycol, the most commonly used in the art is boiling off the water. However, when the water is boiled off, contaminants may be left behind in the monoethylene glycol stream. These contaminants may be removed in a purification process. Some commonly used methods in the art of monoethylene glycol purification are two-step processes where divalent ions are knocked out of the solution with a strong alkali and a solid-liquid separation unit (e.g. a filter press) and then the solution is distilled or vacuum flashed to recover the monoethylene glycol.

These recovery and purification methods may be problematic for several reasons: (1) they may allow for the precipitation of solids in distillation, vacuum flash units or precipitation vessels, (2) they may require a second distillation or vacuum flash step of the monoethylene glycol before the monoethylene glycol can be recycled and (3) they may require solid handling. These methods may also have high energy requirements, costs, and footprints and may require chemical consumption.

An improved method for recovering monoethylene glycol or other alcohols is discussed in PCT/US2014/039667, the entirety of which is hereby incorporated by reference. Briefly, in certain embodiments, the processes discussed therein involve recovering alcohol from an aqueous stream by adding solvent to the aqueous stream, extracting the alcohol and solvent from the aqueous stream using liquid-liquid extraction, and recovering the alcohol and/or the solvent utilizing a using distillation process.

One potential disadvantage of using such a system is that salt from the aqueous stream may accumulate in the glycol stream. After a certain amount of time, saturation level of the salt may reached and the salt may begin to precipitate out of solution. This may lead to scaling on equipment and may cause very costly shut downs of the production system.

There are several processes for removing salt from glycol/water mixtures. In one process the glycol/water mixture is completely flashed off and the remaining salt is disposed of. In another process, the glycol/water stream is mixed with caustic to increase pH. Bivalent ions may then precipitate and be removed in a settler. The bottom product of the settler may then be sent to a centrifuge for concentration and the precipitated salts are disposed of. These processes, however, require solid handling, can be prone to scaling and require a lot of maintenance.

It is desirable to develop a method of recovering alcohols from aqueous solutions comprising salt that do not suffer from the precipitation of salts.

### SUMMARY

The present disclosure relates generally to methods of recovering alcohols from aqueous solutions. More specifically, in certain embodiments, the present disclosure relates to solvents useful for extracting alcohols from aqueous solutions and associated methods.

In one embodiment, the present disclosure provides a method of recovering an alcohol from an aqueous stream comprising: providing an aqueous stream comprising an alcohol; extracting at least a portion of the alcohol from the aqueous stream with a solvent to form an extracted solvent stream; extracting at least a portion of the alcohol from the extracted solvent stream to form an extracted aqueous stream; and recovering at least a portion of the alcohol from the extracted aqueous stream.

In another embodiment, the present disclosure provides method of recovering an alcohol from an aqueous stream comprising: providing an aqueous stream comprising water and an alcohol; providing a solvent stream; combining the solvent stream and the aqueous stream to form an extracted solvent stream; providing a water stream; combining the extracted solvent stream with the water stream to form an extracted aqueous stream; and recovering at least a portion of the alcohol from the extracted aqueous stream.

In another embodiment, the present disclosure provides a system for extracting an alcohol from an aqueous product stream comprising: a first extraction unit, a second extraction unit, and distillation unit.

### BRIEF DESCRIPTION OF THE DRAWINGS

A more complete and thorough understanding of the present embodiments and advantages thereof may be acquired by referring to the following description taken in conjunction with the accompanying drawings.

FIG. 1 is a process flow diagram in accordance with certain embodiments of the present disclosure.

The features and advantages of the present disclosure will be readily apparent to those skilled in the art. While numerous changes may be made by those skilled in the art, such changes are within the spirit of the invention.

### DETAILED DESCRIPTION

The present disclosure relates generally to methods of recovering alcohols from aqueous solutions. More specifically, in certain embodiments, the present disclosure relates to solvents useful for extracting alcohols from aqueous solutions and associated methods.

The description that follows includes exemplary apparatuses, methods, techniques, and/or instruction sequences that embody techniques of the inventive subject matter. However, it is understood that the described embodiments may be practiced without these specific details.

The methods and systems described herein are novel approaches on how to prevent the accumulation of salt in glycol separation systems. In certain embodiments, the methods discussed herein rely on extraction and then back extraction followed by distillation. By performing the methods discussed herein, salt may continuously be rejected from a partial stream, thus keeping the salt concentration in the glycol loop at levels sufficient to prevent the precipitation of the salts in the distillation column or other equipment.

There may be several potential advantages of the methods and systems discussed herein over conventional methods. One potential advantage is that the methods discussed herein may prevent the need for solid handling and reduces complexities in the line-up and avoids the use of centrifuges. Another potential advantage is that since the invention removes salts by the means of extraction, the salts can be disposed off with the water phase instead of separate solids handling. Another potential advantage is that as precipitation of salts is prevented, plugging and scaling of equipment is prevented.

In one embodiment, the present disclosure provides a method of recovering an alcohol from an aqueous stream comprising: providing an aqueous stream comprising an alcohol; extracting at least a portion of the alcohol from the aqueous stream with a solvent to form an extracted solvent stream; extracting at least a portion of the alcohol from the extracted solvent stream to form an extracted aqueous stream; and recovering at least a portion of the alcohol from the extracted aqueous stream.

In certain embodiments, the aqueous stream may be any aqueous stream comprising water and an alcohol. In certain embodiments, the alcohol may comprise one or more monohydric alcohols, one or more polyhydric alcohols, or any combination thereof. Examples of suitable polyhydric alcohols include polyols such as monoethylene glycol, diethylene glycol, dipropylene glycol, triethylene glycol, tetraethylene glycol, propylene glycol, dipropylene glycol, butylene glycol, and glycerol.

In certain embodiments, the amount of water present in the aqueous stream may be an amount in the range of from 10 mol % to 99 mol %. In certain embodiments, the amount of water present in the aqueous stream may be an amount in the range of from 20 mol % to 90 mol %. In certain embodiments, the amount of water present in the aqueous stream may be an amount in the range of from 30 mol % to 85 mol %. In certain embodiments, the amount of water present in the aqueous stream may be an amount in the range of from 40 mol % to 80 mol %. In certain embodiments, the amount of water present in the aqueous stream may be an amount in the range of from 50 mol % to 70 mol %. In certain embodiments, the amount of water present in the aqueous stream may be an amount in the range of from 70 mol % to 99 mol %. In certain embodiments, the amount of water present in the aqueous stream may be an amount in the range of from 80 mol % to 99 mol %. In certain embodiments, the amount of water present in the aqueous stream may be an amount in the range of from 90 mol % to 99 mol %.

In certain embodiments, the amount of alcohol present in the aqueous stream may be an amount in the range of from 1 mol % to 90 mol %. In certain embodiments, the amount of alcohol present in the aqueous stream may be an amount in the range of from 10 mol % to 80 mol %. In certain embodiments, the amount of alcohol present in the aqueous stream may be an amount in the range of from 15 mol % to 70 mol %. In certain embodiments, the amount of alcohol present in the aqueous stream may be an amount in the range of from 20 mol % to 60 mol %. In certain embodiments, the amount of alcohol present in the aqueous stream may be an amount in the range of from 30 mol % to 50 mol %. In certain embodiments, the amount of alcohol present in the aqueous stream may be an amount in the range of from 1 mol % to 30 mol %. In certain embodiments, the amount of alcohol present in the aqueous stream may be an amount in the range of from 1 mol % to 20 mol %. In certain embodiments, the amount of alcohol present in the aqueous stream may be an amount in the range of from 1 mol % to 10 mol %.

In certain embodiments, the aqueous stream may further comprise one or more dissolved salts. Examples of salts that may be included in the aqueous stream include sulfates, chlorides, and carbonates of potassium, sodium, magnesium, iron and calcium. In addition, salts that are formed in corrosion processes, salts used as catalysts and salts formed in acid-base neutralization reactions may be present in the aqueous stream.

In certain embodiments, the dissolved salts may be present in the aqueous stream in an amount that is between zero and the salt solubility limit. In certain embodiments, the salt may be present in an amount in the range of from about 0% to about 25% of the salt solubility limit. In other embodiments, the salt may be present in an amount in the range of from about 0.5% to about 20% of the salt solubility limit. In other embodiments, the salt may be present in an amount in the range of from about 1% to about 5% of the salt solubility limit

In certain embodiments, the aqueous stream may comprise a production stream. In certain embodiments the production stream may be a production stream from a subsea oil and/or gas well. In certain embodiments, the production stream may comprise a mixture of oil, gas, water, and an alcohol. In other embodiments, the aqueous stream may comprise an aqueous stream separated from a production stream.

In certain embodiments, the amount of water present in the production stream may be an amount in the range of from 0% to about 50% by weight of the production stream. In other embodiments, the amount of water present in the production stream may be in an amount in the range of from about 5% to about 50% by weight of the production stream. In other embodiments, the water may be present in the production stream in an amount in the range of from about 10% to about 25% by weight of the production stream.

In certain embodiments, the oil and gas may be present in the production stream in an amount that is between the minimum and maximum solubility of oil and gas in the production stream at given conditions. In certain embodiments, the amount of oil and gas present in the production stream may be an amount in the range of from about 0 mol % to 25 mol %. In other embodiments, the amount of oil and gas present in the production stream may be an amount in the range of from about 0.5 mol % to about 15 mol %. In other embodiments, the amount of oil and gas present in the production stream may be an amount in the range of from about 1 mol % to about 5 mol %.

In certain embodiments, the dissolved salts may be present in the production stream in an amount that is between zero and the salt solubility limit. In certain embodiments, the salt may be present in an amount in the range of from about 0% to about 25% of the salt solubility limit. In other embodiments, the salt may be present in an amount in the range of from about 0.5% to about 20% of the salt solubility limit. In other embodiments, the salt may be present in an amount in the range of from about 1% to about 5% of the salt solubility limit.

In certain embodiments, the production stream may further comprise one or more hydrate forming gases, gases in dense phase, or hydrate forming liquid hydrocarbons. Examples of hydrate forming gases that may be in the production stream include methane, ethane, propane, butane, carbon dioxide and hydrogen sulfide. In certain embodiments, the amount of hydrate forming gas in the production stream, or the amount of hydrate forming liquid hydrocarbon, may be from 0 mol % to 100 mol % based upon the amount of water in the production stream, and typically may be from 25 mol % to 100 mol %.

In certain embodiments, the alcohol may be present in, or added to, the production stream in an amount sufficient to prevent the formation of hydrates in the production stream as it is transported from the subsea oil and gas well. The amount of alcohol necessary to prevent the formation of hydrates may depend on several factors including the pressure of the production stream, the temperature of the production stream, the water content of the production stream, the salinity of the production stream, the concentration of any additives injected in the production stream, and the concentration of hydrate forming gas in the production stream.

A person of ordinary skill in the art would be able to determine a sufficient amount of alcohol to include in the production stream to prevent the formation of hydrates. For example, in certain embodiments the amount of alcohol present in the production stream may be in an amount from 0 mol % to 50 mol % of the water present in the production stream. In other embodiments, the amount of alcohol present in the production stream may be in an amount from 10 mol % to 25 mol % of the water present in the production stream.

In certain embodiments, providing an aqueous stream comprising an alcohol may further comprise separating the aqueous stream into a first portion and a second portion. In certain embodiments, the first portion may comprise up to 50% of the aqueous stream. In other embodiments, the first portion may comprise between 5% and 40% of the aqueous stream. In other embodiments, the first portion may comprise between 10% and 30% of the aqueous stream.

In certain embodiments, extracting at least a portion of the alcohol from the aqueous stream with a solvent to form an extracted solvent stream may comprise extracting at least a portion of the alcohol from the first portion of the aqueous stream. In certain embodiments, extracting at least a portion of the alcohol from the aqueous stream with a solvent to form an extracted solvent stream may further comprise forming a spent aqueous stream.

In certain embodiments, extracting the alcohol from the aqueous stream, or the first portion thereof, with a solvent to form an extracted solvent stream may comprise combining the aqueous stream, or the first portion thereof, with a solvent or a solvent stream. In certain embodiments, the aqueous stream, or the first portion thereof, may be combined with a solvent or a solvent stream within an extraction unit or mixer/settler arrangement. In certain embodiments, extracting at least a portion of the alcohol from the aqueous stream with a solvent to form an extracted solvent stream may further comprise forming a spent aqueous stream.

In certain embodiments, the solvent stream may comprise a solvent and water. In certain embodiments, the amount of solvent present in the solvent stream may be an amount in the range of from 50 mol % to 99 mol %. In certain embodiments, the amount of solvent present in the solvent stream may be an amount in the range of from 60 mol % to 97.5 mol %. In certain embodiments, the amount of solvent present in the solvent stream may be an amount in the range of from 70 mol % to 95 mol %. In certain embodiments, the amount of solvent present in the solvent stream may be an amount in the range of from 80 mol % to 90 mol %. In certain embodiments, the amount of solvent present in the solvent stream may be an amount in the range of from 90 mol % to 99 mol %. In certain embodiments, the amount of solvent present in the solvent stream may be an amount in the range of from 95 mol % to 99 mol %. In certain embodiments, the concentration of solvent in the solvent stream may depend upon the type of solvent used, the temperature of the solvent stream, and the particular extraction equipment used.

In certain embodiments, the amount of water present in the solvent stream may be an amount in the range of from 0.01 mol % to 50 mol %. In certain embodiments, the amount of water present in the solvent stream may be an amount in the range of from 2.5 mol % to 40 mol %. In certain embodiments, the amount of water present in the solvent stream may be an amount in the range of from 5 mol % to 30 mol %. In certain embodiments, the amount of water present in the solvent stream may be an amount in the range of from 10 mol % to 20 mol %. In certain embodiments, the amount of water present in the solvent stream may be an amount in the range of from 1 mol % to 10 mol %. In certain embodiments, the amount of water present in the solvent stream may be an amount in the range of from 1 mol % to 5 mol %. In certain embodiments, the amount of water present in the solvent stream may be an amount in the range of from 0.01 mol % to 2.5 mol %.

In certain embodiments, the solvent stream may further comprise an alcohol. In certain embodiments, the alcohol may comprise any alcohol discussed above or any combination thereof. In certain embodiments, the amount of alcohol present in the solvent stream may be an amount in the range of from 0.01 mol % to 5 mol %. In certain embodiments, the amount of alcohol present in the solvent stream may be an amount in the range of from 0.05 mol % to 2.5 mol %. In certain embodiments, the amount of alcohol present in the solvent stream may be an amount in the range of from 0.1 mol % to 1 mol %. In certain embodiments, the amount of alcohol present in the solvent stream may be an amount in the range of from 0.05 mol % to 0.5 mol %.

In certain embodiments, the solvent may comprise a solvent that is not fully miscible with water. In certain embodiments the solvent may comprise a solvent that has a higher affinity for alcohol than water. In certain embodiments, the solvent may comprise an amine solvent. In certain embodiments, the solvent may comprise a tertiary amine solvent. In certain embodiments, the solvent may comprise dimethylcyclohexylamine, methylcyclohexylamine, methyl piperidine, triethylamine, tripropylamine, or any combination thereof.

In certain embodiments, the extraction unit may comprise of a multistage extraction column. In certain embodiments the column may be equipped with internals such as trays, structured packing or moving internals in order to ensure increase the interface area to facilitate mass transfer between the phases. In other embodiments the extraction equipment may be a single mixer or settler or several serial mixers and/or settlers.

In certain embodiments, the extraction unit may be operated at a temperature in the range of from 15° C. to 90° C. In certain embodiments the extraction unit may be operated at a temperature in the range of from 20° C. to 80° C. In other embodiments, the extraction unit may be operated at a temperature in the range of from 50° C. to 70° C.

In certain embodiments, the alcohol present in the aqueous stream, or first portion thereof, may be extracted from the aqueous stream, or first portion thereof. In certain embodiments, a solvent phase may be formed within the extraction unit, wherein the solvent phase comprises a major portion of the alcohol present in the aqueous stream, or first portion thereof, and minor portions of the water and salt present in the aqueous stream, or first portion thereof. In certain embodiments, an aqueous phase may be formed within the extraction unit, wherein the aqueous phase comprises a minor portion of the alcohol present in the aqueous stream, or first portion thereof, and major portions of the water and salt present in the aqueous stream, or first portion thereof.

In certain embodiments, the aqueous phase may leave the extraction unit as a spent aqueous stream. In certain embodiments, the spent aqueous stream may comprise a major portion of the water from the aqueous stream, or first portion thereof. In certain embodiments, the amount of water present in the spent aqueous stream may be an amount in the range of from 50 mol % to 99 mol %. In certain embodiments, the amount of water present in the spent aqueous stream may be an amount in the range of from 60 mol % to 97.5 mol %. In certain embodiments, the amount of water present in the spent aqueous stream may be an amount in the range of from 70 mol % to 95 mol %. In certain embodiments, the amount of water present in the spent aqueous stream may be an amount in the range of from 80 mol % to 90 mol %. In certain embodiments, the amount of water present in the spent aqueous stream may be an amount in the range of from 90 mol % to 99 mol %. In certain embodiments, the amount of water present in the spent aqueous stream may be an amount in the range of from 95 mol % to 99 mol %.

In certain embodiments, the spent aqueous stream may further comprise a minor portion of the solvent from the solvent stream. In certain embodiments, the amount of solvent present in the spent aqueous stream may be an amount in the range of from 0.01 mol % to 10 mol %. In certain embodiments, the amount of solvent present in the spent aqueous stream may be an amount in the range of from 0.1 mol % to 5 mol %. In certain embodiments, the amount of solvent present in the spent aqueous stream may be an amount in the range of from 1 mol % to 2.5 mol %. In certain embodiments, the amount of solvent present in the spent aqueous stream may be an amount in the range of from 0.01 mol % to 1 mol %. In certain embodiments, the amount of solvent present in the spent aqueous stream may be an amount in the range of from 0.1 mol % to 1 mol %. In certain embodiments, the amount of solvent present in the spent aqueous stream may be an amount in the range of from 2.5 mol % to 10 mol %.

In certain embodiments, the spent aqueous stream may further comprise a minor portion of the alcohol from the aqueous stream, or first portion thereof. In certain embodiments, the amount of alcohol present in the spent aqueous stream may be an amount in the range of from 0.01 mol % to 10 mol %. In certain embodiments, the amount of alcohol present in the spent aqueous stream may be an amount in the range of from 0.1 mol % to 5 mol %. In certain embodiments, the amount of alcohol present in the spent aqueous stream may be an amount in the range of from 1 mol % to 2.5 mol %. In certain embodiments, the amount of alcohol present in the spent aqueous stream may be an amount in the range of from 0.01 mol % to 1 mol %. In certain embodiments, the amount of alcohol present in the spent aqueous stream may be an amount in the range of from 0.1 mol % to 1 mol %. In certain embodiments, the amount of alcohol present in the spent aqueous stream may be an amount in the range of from 2.5 mol % to 10 mol %.

In certain embodiments, the spent aqueous stream may further comprise a major portion of the salt present in the aqueous stream, or first portion thereof. In certain embodiments, the spent aqueous stream may comprise 60% to 100% of the salt present in the aqueous stream or first portion thereof. In certain embodiments, the spent aqueous stream may comprise 70% to 100% of the salt present in the aqueous stream or first portion thereof. In certain embodiments, the spent aqueous stream may comprise 80% to 100% of the salt present in the aqueous stream or first portion thereof. In certain embodiments, the spent aqueous stream may comprise 90% to 100% of the salt present in the aqueous stream or first portion thereof. In certain embodiments, the spent aqueous stream may comprise 95% to 100% of the salt present in the aqueous stream or first portion thereof. In certain embodiments, the spent aqueous stream may comprise 97.5% to 100% of the salt present in the aqueous stream or first portion thereof. In certain embodiments, the spent aqueous stream may comprise 99% to 100% of the salt present in the aqueous stream or first portion thereof. In certain embodiments, the spent aqueous stream may comprise 99.9% to 100% of the salt present in the aqueous stream or first portion thereof.

In certain embodiments, the spent aqueous stream may be disposed of. In other embodiments the solvent in the spent aqueous stream may be separated from the spent aqueous stream in a stripper or flash drum. In other embodiments the spent aqueous stream may be sent to a water purification unit, such as a biological or chemical water treatment plant.

In certain embodiments, the solvent phase may leave the extraction unit as an extracted solvent stream. In certain embodiments, the extracted solvent stream may comprise a minor portion of the water from the aqueous stream, or first portion thereof. In certain embodiments, the amount of water present in the extracted solvent stream may be an amount in the range of from 1 mol % to 50 mol %. In certain embodiments, the amount of water present in the extracted solvent stream may be an amount in the range of from 2.5 mol % to 40 mol %. In certain embodiments, the amount of water present in the spent extracted solvent stream may be an amount in the range of from 5 mol % to 30 mol %. In certain embodiments, the amount of water present in the extracted solvent stream may be an amount in the range of from 10 mol % to 20 mol %. In certain embodiments, the amount of water present in the extracted solvent stream may be an amount in the range of from 1 mol % to 10 mol %. In certain embodiments, the amount of water present in the extracted solvent stream may be an amount in the range of from 1 mol % to 5 mol %.

In certain embodiments, the extracted solvent stream may further comprise a major portion of the solvent from the solvent stream. In certain embodiments, the amount of solvent present in the extracted solvent stream may be an amount in the range of from 50 mol % to 99 mol %. In certain embodiments, the amount of solvent present in the extracted solvent stream may be an amount in the range of from 60 mol % to 90 mol %. In certain embodiments, the amount of solvent present in the spent aqueous stream may be an amount in the range of from 70 mol % to 80 mol %. In certain embodiments, the amount of solvent present in the extracted solvent stream may be an amount in the range of from 60 mol % to 70 mol %. In certain embodiments, the amount of solvent present in the extracted solvent stream may be an amount in the range of from 70 mol % to 90 mol %. In certain embodiments, the amount of solvent present in the extracted solvent stream may be an amount in the range of from 80 mol % to 99 mol %.

In certain embodiments, the extracted solvent stream may further comprise a major portion of the alcohol from the aqueous stream, or first portion thereof. In certain embodiments, the amount of alcohol present in the extracted solvent stream may be an amount in the range of from 1 mol % to 50 mol %. In certain embodiments, the amount of alcohol present in the extracted solvent stream may be an amount in the range of from 10 mol % to 40 mol %. In certain embodiments, the amount of alcohol present in the extracted solvent stream may be an amount in the range of from 10 mol % to 30 mol %. In certain embodiments, the amount of alcohol present in the extracted solvent stream may be an amount in the range of from 20 mol % to 30 mol %. In certain embodiments, the amount of alcohol present in the extracted solvent stream may be an amount in the range of from 30 mol % to 40 mol %. In certain embodiments, the amount of alcohol present in the extracted solvent stream may be an amount in the range of from 1 mol % to 20 mol %. In certain embodiments, the amount of alcohol present in the extracted solvent stream may be an amount in the range of from 20 mol % to 50 mol %.

In certain embodiments, the extracted solvent stream may further comprise a minor portion of the salt present in the aqueous stream, or first portion thereof. In certain embodiments, the extracted solvent stream may comprise 0% to 40% of the salt present in the aqueous stream or first portion thereof. In certain embodiments, the extracted solvent stream may comprise 0% to 30% of the salt present in the aqueous stream or first portion thereof. In certain embodiments, the extracted solvent stream may comprise 0% to 20% of the salt present in the aqueous stream or first portion thereof. In certain embodiments, the extracted solvent stream may comprise 0% to 10% of the salt present in the aqueous stream or first portion thereof. In certain embodiments, the extracted solvent stream may comprise 0% to 5% of the salt present in the aqueous stream or first portion thereof. In certain embodiments, the extracted solvent stream may comprise 0% to 2.5% of the salt present in the aqueous stream or first portion thereof. In certain embodiments, the extracted solvent stream may comprise 0% to 1% of the salt present in the aqueous stream or first portion thereof. In certain embodiments, the extracted solvent stream may comprise 0.1% to 1% of the salt present in the aqueous stream or first portion thereof.

In certain embodiments, extracting at least a portion of the alcohol from the extracted solvent stream to form an extracted aqueous stream may comprise extracting at least a portion of the alcohol from the extracted solvent stream. In certain embodiments, extracting at least a portion of the alcohol from the extracted solvent stream to form an extracted aqueous stream may further comprise forming a spent solvent stream.

In certain embodiments, extracting at least a portion of the alcohol from the extracted solvent stream to form an extracted aqueous stream may comprise combining the extracted solvent stream with water or water stream. In certain embodiments, the extracted solvent stream may be combined with water or a water stream within an extraction unit. In certain embodiments, extracting at least a portion of the alcohol from the extracted solvent stream with water to form an extracted aqueous stream may further comprise forming a spent solvent stream.

In certain embodiments, the water stream may comprise water and traces of solvent and alcohol. In certain embodiments the traces of solvent and alcohol may be below 1%.

In certain embodiments, the extraction unit may comprise an extraction column. In certain embodiments, the extraction column may be equipped with trays. In other embodiments, the extraction column may be equipped with structured packing or rotating internals. In other embodiments, the extraction unit may comprise one or more mixers and/or settlers. In certain embodiments, the extraction unit may be operated at a temperature in the range of from 100° C. to about 150° C.

In certain embodiments, the alcohol present in the extracted solvent stream may be extracted from the extracted solvent stream. In certain embodiments, a solvent phase may be formed within the extraction unit, wherein the solvent phase comprises a major portion of the solvent in the extracted solvent stream and a minor portion of the alcohol from the extracted solvent stream and the water from the water stream. In certain embodiments, an aqueous phase may be formed within the extraction unit, wherein the aqueous phase comprises major portions of the alcohol present in the extracted solvent stream and water from the water stream and a minor portion of the solvent from the extracted solvent stream.

In certain embodiments, the aqueous phase may leave the extraction unit as an extracted aqueous stream. In certain embodiments, the extracted aqueous stream may comprise a major portion of the water from the water stream. In certain embodiments, the amount of water present in the extracted aqueous stream may be an amount in the range of from 50 mol % to 90 mol %. In certain embodiments, the amount of water present in the extracted aqueous stream may be an amount in the range of from 60 mol % to 80 mol %. In certain embodiments, the amount of water present in the extracted aqueous stream may be an amount in the range of from 70 mol % to 80 mol %. In certain embodiments, the amount of water present in the extracted aqueous stream may be an amount in the range of from 80 mol % to 90 mol %. In certain embodiments, the amount of water present in the extracted aqueous stream may be an amount in the range of from 50 mol % to 80 mol %. In certain embodiments, the amount of water present in the extracted aqueous stream may be an amount in the range of from 60 mol % to 70 mol %.

In certain embodiments, the extracted aqueous stream may comprise a minor portion of the solvent from the extracted solvent stream. In certain embodiments, the amount of solvent present in the extracted aqueous stream may be an amount in the range of from 0.01 mol % to 10 mol %. In certain embodiments, the amount of solvent present in the extracted aqueous stream may be an amount in the range of from 0.1 mol % to 5 mol %. In certain embodiments, the amount of solvent present in the extracted aqueous stream may be an amount in the range of from 1 mol % to 2.5 mol %. In certain embodiments, the amount of solvent present in the extracted aqueous stream may be an amount in the range of from 0.01 mol % to 1 mol %. In certain embodiments, the amount of solvent present in the extracted aqueous stream may be an amount in the range of from 0.1 mol % to 1 mol %. In certain embodiments, the amount of solvent present in the extracted aqueous stream may be an amount in the range of from 2.5 mol % to 10 mol %.

In certain embodiments, the extracted aqueous stream may comprise a major portion of the alcohol from the extracted solvent stream. In certain embodiments, the amount of alcohol present in the extracted aqueous stream may be an amount in the range of from 10 mol % to 50 mol %. In certain embodiments, the amount of alcohol present in the extracted aqueous stream may be an amount in the range of from 20 mol % to 50 mol %. In certain embodiments, the amount of alcohol present in the extracted aqueous stream may be an amount in the range of from 20 mol % to 40 mol %. In certain embodiments, the amount of alcohol present in the extracted aqueous stream may be an amount in the range of from 30 mol % to 50 mol %. In certain embodiments, the amount of alcohol present in the extracted aqueous stream may be an amount in the range of from 30 mol % to 40 mol %. In certain embodiments, the amount of alcohol present in the extracted aqueous stream may be an amount in the range of from 40 mol % to 50 mol %.

In certain embodiments, the extracted aqueous stream may further comprise a major portion of the salt present in the extracted solvent stream. In certain embodiments, the extracted aqueous stream may comprise 60% to 100% of the salt present in the extracted solvent stream. In certain embodiments, the extracted aqueous stream may comprise 70% to 100% of the salt present in the extracted solvent stream. In certain embodiments, the extracted aqueous stream may comprise 80% to 100% of the salt present in the extracted solvent stream. In certain embodiments, the extracted aqueous stream may comprise 90% to 100% of the salt present in the extracted solvent stream. In other embodiments, the extracted aqueous stream may comprise a negligible amount of salt.

In certain embodiments, the solvent phase may leave the extraction unit as a spent solvent stream. In certain embodiments, the spent solvent stream may comprise a major portion of the solvent from the extracted solvent stream. In certain embodiments, the amount of solvent present in the spent solvent stream may be an amount in the range of from 80 mol % to 99 mol %. In certain embodiments, the amount of solvent present in the spent solvent stream may be an amount in the range of from 80 mol % to 95 mol %. In certain embodiments, the amount of solvent present in the spent solvent stream may be an amount in the range of from 90 mol % to 99 mol %. In certain embodiments, the amount of solvent present in the spent solvent stream may be an amount in the range of from 85 mol % to 90 mol %. In certain embodiments, the amount of solvent present in the spent solvent stream may be an amount in the range of from 90 mol % to 95 mol %. In certain embodiments, the amount of solvent present in the spent solvent stream may be an amount in the range of from 85 mol % to 90 mol %.

In certain embodiments, the spent solvent stream may comprise a minor portion of the water from the extracted solvent stream. In certain embodiments, the amount of water present in the spent solvent stream may be an amount in the range of from 0.01 mol % to 20 mol %. In certain embodiments, the amount of water present in the spent solvent stream may be an amount in the range of from 0.1 mol % to 10 mol %. In certain embodiments, the amount of water present in the spent solvent stream may be an amount in the range of from 1 mol % to 10 mol %. In certain embodiments, the amount of water present in the spent solvent stream may be an amount in the range of from 1 mol % to 5 mol %. In certain embodiments, the amount of water present in the spent solvent stream may be an amount in the range of from 2.5 mol % to 5 mol %. In certain embodiments, the amount of water present in the spent solvent stream may be an amount in the range of from 2.5 mol % to 10 mol %.

In certain embodiments, the spent solvent stream may comprise a minor portion of the alcohol from the extracted solvent stream. In certain embodiments, the amount of alcohol present in the spent solvent stream may be an amount in the range of from 0 mol % to 5 mol %. In certain embodiments, the amount of alcohol present in the spent solvent stream may be an amount in the range of from 0.01 mol % to 2.5 mol %. In certain embodiments, the amount of alcohol present in the spent solvent stream may be an amount in the range of from 0.1 mol % to 1 mol %. In certain embodiments, the amount of alcohol present in the spent solvent stream may be an amount in the range of from 0.25 mol % to 0.5 mol %.

In certain embodiments, the extracted aqueous stream may comprise a minor portion of the salt present in the extracted solvent stream. In certain embodiments, the spent solvent stream may comprise 0% to 40% of the salt present in the extracted solvent stream. In certain embodiments, the spent solvent stream may comprise 0% to 30% of the salt present in the extracted solvent stream. In certain embodiments, the spent solvent stream may comprise 0% to 20% of the salt present in the extracted solvent stream. In certain embodiments, the spent solvent stream may comprise 0% to 10% of the salt present in the extracted solvent stream. In other embodiments, the extracted aqueous stream may comprise a negligible amount of salt.

In certain embodiments, the spent solvent stream or a portion thereof may be recycled as the solvent stream.

In certain embodiments, recovering at least a portion of the alcohol from the extracted aqueous stream may comprise recovering at least a portion of the alcohol from the extracted aqueous stream and the second portion of the aqueous stream. In certain embodiments, recovering at least a portion of the alcohol from the extracted aqueous stream may comprise combining the extracted aqueous stream with the second portion of the aqueous stream to form a combined aqueous stream and recovering at least a portion of the alcohol from the combined aqueous stream. In certain embodiments, recovering at least a portion of the alcohol from extracted aqueous stream may be accomplished by performing distillation.

In certain embodiments, recovering the alcohol from the extracted aqueous stream, the second portion of the aqueous stream, and/or the combined aqueous stream, may comprise forming a recovered alcohol stream. In certain embodiments, recovering the alcohol from the extracted aqueous stream, the second portion of the aqueous stream, and/or the combined aqueous stream, may comprise forming a recovered aqueous stream. In certain embodiments, the step of recovering the alcohol from the extracted aqueous stream, the second portion of the aqueous stream, and/or the combined aqueous stream may be performed within a distillation unit.

In certain embodiments, the distillation unit may comprise a rectification column. In certain embodiments, the distillation column may be equipped with a reboiler. In certain embodiments, the reboiler may be a kettle type or a thermosyphon type reboiler. In certain embodiments, the distillation unit may be operated at a temperature in the range of from 90° C. to 160° C.

In certain embodiments, a major portion of the alcohol, along with a minor portion of the water, present in the extracted aqueous stream, the second portion of the aqueous stream, and/or the combined aqueous stream may leave the distillation unit as a recovered alcohol stream.

In certain embodiments, the amount of water present in the recovered alcohol stream may be an amount in the range of from 0 mol % to 50 mol %. In certain embodiments, the amount of water present in the recovered alcohol stream may be an amount in the range of from 0 mol % to 40 mol %. In certain embodiments, the amount of water present in the recovered alcohol stream may be an amount in the range of from 5 mol % to 40 mol %. In certain embodiments, the amount of water present in the recovered alcohol stream may be an amount in the range of from 10 mol % to 40 mol %. In certain embodiments, the amount of water present in the recovered alcohol stream may be an amount in the range of from 20 mol % to 40 mol %. In certain embodiments, the amount of water present in the recovered alcohol stream may be an amount in the range of from 30 mol % to 40 mol %.

In certain embodiments, the amount of solvent present in the recovered alcohol stream may be an amount in the range of from 0.01 mol % to 5 mol %. In certain embodiments, the amount of solvent present in the recovered alcohol stream may be an amount in the range of from 0.1 mol % to 2.5 mol %. In certain embodiments, the amount of solvent present in the recovered alcohol stream may be an amount in the range of from 0.1 mol % to 1 mol %. In certain embodiments, the amount of solvent present in the recovered alcohol stream may be an amount in the range of from 0.01 mol % to 0.5 mol %. In certain embodiments, the amount of solvent present in the recovered alcohol stream may be an amount in the range of from 0.5 mol % to 1 mol %. In certain embodiments, the amount of solvent present in the recovered alcohol stream may be an amount in the range of from 0.25 mol % to 0.5 mol %.

In certain embodiments, the amount of alcohol present in the recovered alcohol stream may be an amount in the range of from 50 mol % to 99 mol %. In certain embodiments, the amount of alcohol present in the recovered alcohol stream may be an amount in the range of from 60 mol % to 99 mol %. In certain embodiments, the amount of alcohol present in the recovered alcohol stream may be an amount in the range of from 70 mol % to 95 mol %. In certain embodiments, the amount of alcohol present in the recovered alcohol stream may be an amount in the range of from 80 mol % to 90 mol %. In certain embodiments, the amount of alcohol present in the recovered alcohol stream may be an amount in the range of from 85 mol % to 95 mol %. In certain embodiments, the amount of alcohol present in the recovered alcohol stream may be an amount in the range of from 50 mol % to 80 mol %.

In certain embodiments, a major portion of the water, along with a minor portion of the alcohol, present in the extracted aqueous stream, the second portion of the aqueous stream, and/or the combined aqueous stream may leave the distillation unit as a recovered aqueous stream.

In certain embodiments, the amount of water present in the recovered aqueous stream may be an amount in the range of from 80 mol % to 99.9 mol %. In certain embodiments, the amount of water present in the recovered aqueous stream may be an amount in the range of from 85 mol % to 99.9 mol %. In certain embodiments, the amount of water present in the recovered aqueous stream may be an amount in the range of from 90 mol % to 95 mol %. In certain embodiments, the amount of water present in the recovered aqueous stream may be an amount in the range of from 90 mol % to 99.9 mol %. In certain embodiments, the amount of water present in the recovered aqueous stream may be an amount in the range of from 95 mol % to 99.9 mol %. In certain embodiments, the amount of water present in the recovered aqueous stream may be an amount in the range of from 85 mol % to 95 mol %. In certain embodiments, the amount of water present in the recovered aqueous stream may be an amount in the range of from 85 mol % to 90 mol %.

In certain embodiments, the amount of solvent present in the recovered aqueous stream may be an amount in the range of from 0.01 mol % to 10 mol %. In certain embodiments, the amount of solvent present in the recovered aqueous stream may be an amount in the range of from 0.01 mol % to 5 mol %. In certain embodiments, the amount of solvent present in the recovered aqueous stream may be an amount in the range of from 0.1 mol % to 2.5 mol %. In certain embodiments, the amount of solvent present in the recovered aqueous stream may be an amount in the range of from 0.01 mol % to 1 mol %. In certain embodiments, the amount of solvent present in the recovered aqueous stream may be an amount in the range of from 0.5 mol % to 1 mol %. In certain embodiments, the amount of solvent present in the recovered aqueous stream may be an amount in the range of from 0.25 mol % to 0.5 mol %.

In certain embodiments, the amount of alcohol present in the recovered aqueous stream may be an amount in the range of from 0.01 mol % to 10 mol %. In certain embodiments, the amount of alcohol present in the recovered aqueous stream may be an amount in the range of from 0.01 mol % to 5 mol %. In certain embodiments, the amount of alcohol present in the recovered aqueous stream may be an amount in the range of from 0.1 mol % to 2.5 mol %. In certain embodiments, the amount of alcohol present in the recovered aqueous stream may be an amount in the range of from 0.01 mol % to 1 mol %. In certain embodiments, the amount of alcohol present in the recovered aqueous stream may be an amount in the range of from 0.5 mol % to 1 mol %. In certain embodiments, the amount of alcohol present in the recovered aqueous stream may be an amount in the range of from 0.25 mol % to 0.5 mol %.

In certain embodiments, the recovered aqueous stream may be disposed of. In certain embodiments, the recovered aqueous stream, or a portion thereof, may be recycled as the water stream.

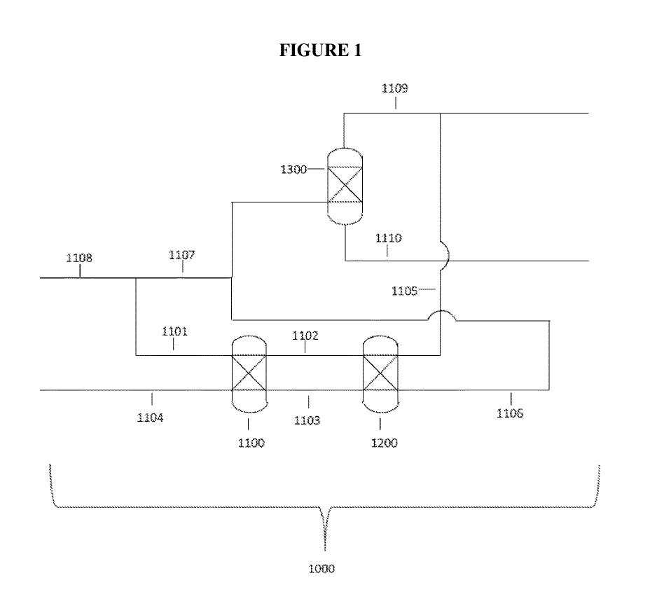

In another embodiment, the present disclosure provides a system for extracting an alcohol from an aqueous product stream comprising: a first extraction unit, a second extraction unit, and distillation unit. Such a system is illustrated in FIG. 1.

Referring now to FIG. 1, FIG. 1 illustrates a glycol removal system 1000 comprising a first extraction unit 1100, a second extraction unit 1200, and a distillation unit 1300.

In certain embodiments, first extraction unit 1100 may comprise any extraction unit discussed above. In certain embodiments, second extraction unit 1200 may comprise any unit discussed above. In certain embodiments, distillation unit 1300 may comprise any distillation unit discussed above.

With respect to first extraction unit 1100, as can be seen in FIG. 1, aqueous stream 1101 and solvent stream 1102 may be supplied to first extraction unit 1100. In certain embodiments, aqueous stream 1101 may comprise any aqueous stream discussed above. In certain embodiments, solvent stream 1102 may comprise any solvent stream discussed above.

In certain embodiments, alcohol may be extracted from aqueous stream 1101 within first extraction unit 1100 by solvent stream 1102 thereby forming extracted solvent stream 1103 and spent aqueous stream 1104.

In certain embodiments, spent aqueous stream 1104 may comprise any spent aqueous stream discussed above. In certain embodiments, extracted solvent stream 1103 may comprise any extracted solvent stream discussed above.

With respect to second extraction unit 1200, as can be seen in FIG. 1, extracted solvent stream 1103 and water stream 1105 may be supplied to second extraction unit 1200. In certain embodiments, water stream 1105 may comprise any water stream discussed above.

In certain embodiments, alcohol may be extracted from extracted solvent stream 1103 by water stream 1105 thereby forming solvent stream 1102 and extracted aqueous stream 1106.

In certain embodiments, extracted aqueous stream 1106 may comprise any extracted aqueous stream discussed above. In certain embodiments, solvent stream 1102 may comprise any spent solvent stream discussed above.

With respect to distillation unit 1300, in certain embodiments, not illustrated, extracted aqueous stream 1106 may be supplied to directly to distillation unit 1300. In other embodiments, aqueous stream 1107 may be added to aqueous stream 1106 before it is supplied to distillation unit 1300. In certain embodiments, aqueous stream 1101 may comprise a first portion of aqueous stream 1108 and aqueous stream 1107 may comprise a second portion of aqueous stream 1108. In certain embodiments, aqueous streams 1107 and 1108 may have the same compositional make up of aqueous stream 1101. In certain embodiments, aqueous stream 1107 and 1108 may comprise any aqueous stream discussed above, and any portion thereof.

In certain embodiments, alcohol may be distilled from extracted aqueous stream 1106 within distillation unit 1300 thereby forming recovered aqueous stream 1109 and recovered alcohol stream 1110. In certain embodiments, recovered aqueous stream 1109 may comprise any recovered aqueous stream discussed above. In certain embodiments, recovered alcohol stream 1110 may comprise any recovered alcohol stream discussed above.

In certain embodiments, a portion of recovered aqueous stream 1109 may be recycled as water stream 1105.

While the embodiments are described with reference to various implementations and exploitations, it will be understood that these embodiments are illustrative and that the scope of the inventive subject matter is not limited to them. Many variations, modifications, additions and improvements are possible.

Plural instances may be provided for components, operations or structures described herein as a single instance. In general, structures and functionality presented as separate components in the exemplary configurations may be implemented as a combined structure or component. Similarly, structures and functionality presented as a single component may be implemented as separate components. These and other variations, modifications, additions, and improvements may fall within the scope of the inventive subject matter.

1. A method of recovering an alcohol from an aqueous stream comprising:
providing an aqueous stream comprising an alcohol;
extracting at least a portion of the alcohol from the aqueous stream with a solvent to form an extracted solvent stream;
extracting at least a portion of the alcohol from the extracted solvent stream to form an extracted aqueous stream; and
recovering at least a portion of the alcohol from the extracted aqueous stream.

2. The method of claim 1, wherein the alcohol comprises at least one alcohol selected from the group consisting of: monoethylene glycol, diethylene glycol, dipropylene glycol, triethylene glycol, tetraethylene glycol, propylene glycol, dipropylene glycol, butylene glycol, glycerol, and any combination thereof.

3. The method of claim 1, wherein the aqueous stream comprises an amount of water in the range of from 70 mol % to 99 mol %.

4. The method of claim 1, wherein the aqueous stream comprises an amount of alcohol in the range of from 1 mol % to 30 mol %.

5. The method of claim 1, wherein the aqueous stream comprises an aqueous stream separated from a production stream.

6. The method of claim 1, wherein the solvent comprise at least one solvent selected from the group consisting of: dimethylcyclohexylamine, methylcyclohexylamine, methyl piperidine, triethylamine, and tripropylamine.

7. The method of claim 1, wherein the extracted solvent stream comprises an amount of alcohol in the range of from 10 mol % to 30 mol %.

8. The method of claim 1, wherein the extracted aqueous stream comprises an amount of alcohol in the range of from 10 mol % to 50 mol %.

9. The method of claim 1, wherein the aqueous stream comprises a salt.

10. A method of recovering an alcohol from an aqueous stream comprising:
providing an aqueous stream comprising water and an alcohol;
providing a solvent stream;
combining the solvent stream and the aqueous stream to form an extracted solvent stream;
providing a water stream;
combining the extracted solvent stream with the water stream to form an extracted aqueous stream; and
recovering at least a portion of the alcohol from the extracted aqueous stream.

11. The method of claim 10, wherein the alcohol comprises at least one alcohol selected from the group consisting of: monoethylene glycol, diethylene glycol, dipropylene glycol, triethylene glycol, tetraethylene glycol, propylene glycol, dipropylene glycol, butylene glycol, glycerol, and any combination thereof.

12. The method of claim 10, wherein the aqueous stream comprises an amount of water in the range of from 70 mol % to 99 mol %.

13. The method of claim 10, wherein the aqueous stream comprises an amount of alcohol in the range of from 1 mol % to 30 mol %.

14. The method of claim 10, wherein the aqueous stream comprises an aqueous stream separated from a production stream.

15. The method of claim 10, wherein the solvent comprise at least one solvent selected from the group consisting of: dimethylcyclohexylamine, methylcyclohexylamine, methyl piperidine, triethylamine, and tripropylamine.

16. The method of claim 10, wherein the extracted solvent stream comprises an amount of alcohol in the range of from 10 mol % to 30 mol %.

17. The method of claim 10, wherein the extracted aqueous stream comprises an amount of alcohol in the range of from 10 mol % to 50 mol %.

18. The method of claim 10, wherein the aqueous stream comprises a salt.

19. A system for extracting an alcohol from an aqueous product stream comprising: a first extraction unit, a second extraction unit, and distillation unit.

20. The system of claim 19, wherein an aqueous stream and a solvent stream are supplied to the first extraction unit, an extracted solvent stream and a water stream are supplied to the second extraction unit, and an extracted aqueous stream is supplied to the distillation unit.
